(19) United States
(12) Patent Application Publication
(10) Pub. No.: US 2024/0246046 A1
(43) Pub. Date: Jul. 25, 2024

(54) METHOD FOR PRODUCING ULTRAFINE-BUBBLE-CONTAINING LIQUID AND APPARATUS FOR PRODUCING ULTRAFINE-BUBBLE-CONTAINING LIQUID

(71) Applicant: CANON KABUSHIKI KAISHA, Tokyo (JP)

(72) Inventors: YOHEI MASADA, Tokyo (JP); TAKAHARU AOTANI, Tokyo (JP); IKUO NAKAZAWA, Kanagawa (JP)

(21) Appl. No.: 18/624,370

(22) Filed: Apr. 2, 2024

Related U.S. Application Data
(63) Continuation of application No. PCT/JP2022/030995, filed on Aug. 16, 2022.

(30) Foreign Application Priority Data
Nov. 4, 2021 (JP) 2021-180401

(51) Int. Cl. B01F 31/85; A61K 9/107; A61K 31/7036; B01F 23/232; B01F 25/452; C02F 1/68; C02F 1/74; C02F 103/04

(52) U.S. Cl. CPC B01F 31/85; A61K 9/107; A61K 31/7036; B01F 23/232; B01F 25/4521; C02F 1/685; C02F 1/74; B01F 2215/0431; C02F 2103/04

(57) ABSTRACT

A method for producing an ultrafine-bubble-containing liquid includes: supplying a liquid containing at least one substance selected from a group stated below to a liquid supply region where an ejection orifice forming member in which an ejection orifice with a minute diameter is formed is disposed; and ejecting the liquid from the ejection orifice as a droplet by causing a pressure application unit in contact with the liquid to apply pressure to the liquid supplied to the liquid supply region. The group consists of a thermally denaturing material, inorganic salts, amino acids and their salts, carbohydrates/sugars and their salts, organic polymers and their salts, inorganic polymers and their salts, a dispersion, and an organic liquid.

### CROSS-REFERENCE TO RELATED APPLICATIONS

This application is a Continuation of International Patent Application No. PCT/JP2022/030995, filed Aug. 16, 2022, which claims the benefit of Japanese Patent Application No. 2021-180401, filed Nov. 4, 2021, both of which are hereby incorporated by reference herein in their entirety.

### BACKGROUND OF THE INVENTION

### Field of the Invention

The present disclosure relates to a technique for producing an ultrafine-bubble-containing liquid.

### Background Art

A liquid containing ultrafine bubbles (hereinafter also referred to as “UFBs”), which are less than 1.0 μm in diameter, is characterized in that UFBs do not float to the surface but remain in the liquid, and its usefulness in various fields has been confirmed.

As a technique for producing an UFB-containing liquid, PTL 1 discloses a technique for generating water containing oxygen nanobubbles with a diameter of 10 nm or larger and 500 nm or smaller by generating swirling flows in water having oxygen mixed therein and causing the swirling flows to collide with each other.

Also, PTL 2 describes a UFB producing method characterized by producing UFBs by causing film boiling in a liquid.

### CITATION LIST

### Patent Literature

PTL 1: Japanese Patent Laid-Open No. 2011-200778

PTL 2: Japanese Patent Laid-Open No. 2019-42732

### SUMMARY OF THE INVENTION

In the technique disclosed in PTL 1, the produced liquid contains, along with UFBs, large quantities of air bubbles with large diameters of 1.0 μm or above (microbubbles, milli-bubbles) and the like as well. Because these bubbles float to the surface of water and burst, there is a high possibility that the water cannot hold the bubbles for a long period of time. Also, it has been found that in floating to the surface and bursting, the air bubbles affect the UFBs, reducing UFB concentration. Thus, it is difficult to produce a high-purity UFB-containing liquid which has high concentration and can be stored for a long period of time.

By contrast, the UFB producing method disclosed in PTL 2 can generate a UFB-containing liquid in which the particle sizes of the UFBs are all approximately 100 nm. However, because the technique disclosed in PTL 2 generates UFBs by heating a liquid and causing film boiling therein, there are liquids that are not suited to be used for long-term production of a UFB-containing liquid depending on the composition of the liquid in which to generate UFBs. For example, with a liquid containing a solid component or a metallic salt or with a liquid using an organic solvent, it may be difficult to produce a UFB-containing liquid stably for a long period of time.

In a first aspect of the present invention, there is provided a method for producing an ultrafine-bubble-containing liquid, the method comprising: supplying a liquid containing at least one substance selected from a group stated below to a liquid supply region where an ejection orifice forming member in which an ejection orifice with a minute diameter is formed is disposed; and ejecting the liquid from the ejection orifice as a droplet by causing a pressure application unit in contact with the liquid to apply pressure to the liquid supplied to the liquid supply region, the group consisting of a thermally denaturing material, inorganic salts, amino acids and their salts, carbohydrates/sugars and their salts, organic polymers and their salts, inorganic polymers and their salts, a dispersion, and an organic liquid.

In a second aspect of the present invention, there is provided an apparatus for producing an ultrafine-bubble-containing liquid, the apparatus comprising: an ejection orifice forming member in which an ejection orifice with a minute diameter is formed and which is disposed at a liquid supply region to which a liquid containing at least one substance selected from a group stated below is supplied; and a pressure application unit that causes the liquid to be ejected from the ejection orifice as a droplet by being in contact with the liquid and applying pressure to the liquid supplied to the liquid supply region, the group consisting of a thermally denaturing material, inorganic salts, amino acids and their salts, carbohydrates/sugars and their salts, organic polymers and their salts, inorganic polymers and their salts, a dispersion, and an organic liquid.

Further features of the present invention will become apparent from the following description of exemplary embodiments with reference to the attached drawings.

### BRIEF DESCRIPTION OF THE DRAWINGS

FIG. 1 is a diagram showing an example of a UFB-containing liquid producing apparatus of the present embodiment; and

FIG. 2 is a diagram schematically showing the configuration of an UFB generating device used in the present embodiment.

### DESCRIPTION OF THE EMBODIMENTS

An embodiment of the present disclosure is described in detail below with reference to the drawings attached hereto. Note that the following embodiment is not intended to limit the present invention defined by the scope of claims, and not all the combinations of features described in the present embodiment are essential as solutions provided by the present invention. Also, in the following description, an ultrafine bubble, which is an air bubble (or a bubble) with a diameter of 1.0 μm or smaller, is referred to also as a “UFB,” and an ultrafine-bubble-containing liquid is referred to also as a “UFB-containing liquid.”

### (UFB-Containing Liquid Producing Apparatus)

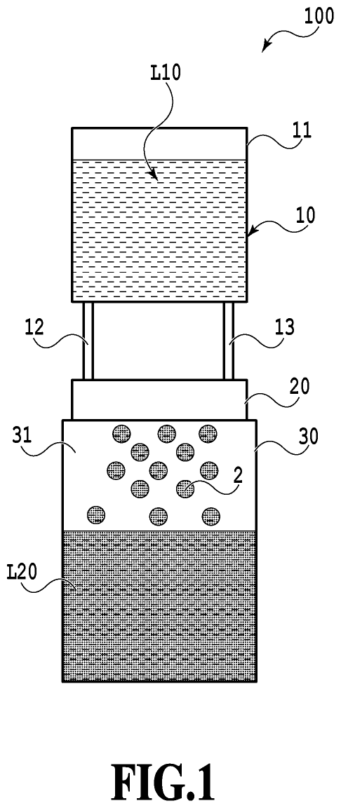

FIG. 1 is a diagram showing an example of a UFB-containing liquid producing apparatus 100 of the present embodiment. The UFB-containing liquid producing apparatus 100 includes a supply unit 10 as a liquid supply unit that supplies a gas-dissolved liquid L10, a UFB generation device 20, and a UFB-containing liquid collection unit (collection unit) 30. The supply unit 10 supplies the gas-dissolved liquid (air-dissolved water) L10 to the UFB generation device 20 via a supply flow channel 12, the gas-dissolved liquid L10 being obtained by dissolving air (atmosphere) in a liquid (water in this example) supplied to a gas dissolving tank 11. The gas-dissolved liquid L10 is generated using, e.g., a bubbling mechanism (a gas dissolution unit) (not shown) that introduces air into the liquid supplied to the gas dissolving tank 11 and agitates the liquid. Although air (atmosphere) is used as an example of gas in the embodiment described, gas to be dissolved in a liquid is not limited to air and may be other type of gas.

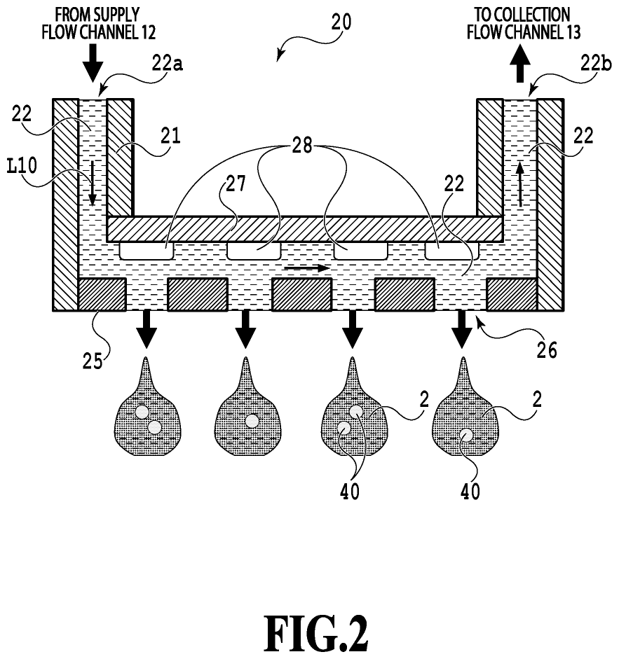

The UFB generation device 20 ejects the air-dissolved water L10 supplied from the supply unit 10 as minute droplets. The UFB generation device 20 is provided above the collection unit 30 and ejects a large number of droplets 2 from ejection orifices 26 (FIG. 2) to be described later to a space region 31 of the collection unit 30. Meanwhile, part of the gas-dissolved liquid L10 supplied to the UFB generation device 20 but not used for the droplet ejection returns back to the supply unit 10 via a collection flow channel 13 by a pump or the like (not shown). In this way, the present embodiment employs a circulation approach where the gas-dissolved liquid L10 is supplied from the supply unit 10 to the UFB generation device and then back to the supply unit 10.

### <UFB Generation Device>

FIG. 2 is a diagram schematically showing the configuration of the UFB generation device 20 used in the present embodiment. The UFB generation device 20 includes a flow-channel forming member 21 forming a flow channel 22 extending from a liquid inflow port 22a to a liquid outflow port 22b. The liquid inflow port 22a is connected to the supply flow channel 12 shown in FIG. 1, and the liquid outflow port 22b is connected to the collection flow channel 13 shown in FIG. 1. Part of the flow-channel forming member 21 is provided with a plate-shaped ejection orifice forming member 25 having a plurality of minute ejection orifices 26 formed therein and an element holding member 27 at a position facing the ejection orifice forming member 25. Note that it is desirable that the ejection orifices 26 with a minute diameter be 100 μm or smaller in diameter. The element holding member 27 is provided with a plurality of piezoelectric elements (pressure application units) 28 facing the ejection orifices 26.

Each piezoelectric element 28 is in contact with the gas-dissolved liquid L10 filling the flow channel 22, which is a liquid supply region, and deforms toward the ejection orifice 26 upon application of a drive voltage from a drive unit (not shown), thereby applying pressure to the gas-dissolved liquid L10 directly. Due to the pressure thus applied, the gas-dissolved liquid L10 filling the flow channel 22 and the ejection orifice 26 is ejected from the ejection orifice 26 in a direction denoted by the arrow as a droplet 2 and is ejected into the collection unit 30.

The droplet 2 ejected from the ejection orifice 26 of the UFB generation device 20 contains UFBs 40 therein. The mechanism of how the droplet 2 contains UFBs is conjectured as follows. In the event where the gas-dissolved liquid L10 is ejected from the ejection orifice 26 with a minute diameter as a droplet 2, a large shear stress acts on the liquid L10 between the liquid L10 and the surrounding wall surface of the ejection orifice 26. The liquid L10 flies as the droplet 2 into the atmosphere immediately after that and is consequently released from the shear stress. In this event, the gas-dissolved liquid L10 undergoes a great pressure change in a short period of time. It is supposed that due to this great pressure change, dissolved gas in the gas-dissolved liquid L10 undergoes a phase change and changes into a gas state, forming the UFBs 40. Further, in the UFB generation device 20 described above, the piezoelectric elements 28 provided in correspondence with the respective ejection orifices 26 cause uniform pressure change in the event of the ejection of the droplets 2, allowing the UFBs 40 to have almost the same size, which is a conceivable reason why uniform UFBs with narrowly distributed particle size can be created.

Note that the number of ejection orifices 26 with a minute diameter formed in the ejection orifice forming member 25 described above is not limited to any particular number. There may be a plurality of them as in the above embodiment, or may be a single one of them. Irrespective of whether there may be one or more than one ejection orifice 26, it is preferable that there be one piezoelectric element for one ejection orifice.

Also, the droplet 2 containing the UFBs 40 is collected by the collection unit 30 shaped as a container and becomes a UFB-containing liquid L20 (see FIG. 1). It is possible to supply this UFB-containing liquid L20 back to the supply unit 10 or another UFB generation device 20 and eject it as droplets again. In other words, ejection and collection of the UFB-containing liquid can be repeated. This can increase a UFB concentration in the UFB-containing liquid.

### <Collection Unit>

FIG. 1 is referred to again. The collection unit 30 is attached below the UFB generation device 20 and collects the droplets 2 containing the UFBs 40. The droplets 2 collected are retained in the collection unit 30 as the UFB-containing liquid L20. As shown in FIG. 1, it is preferable that the collection unit 30 be in close contact with the UFB generation device 20 and structured to prevent intrusion of dust from the outside. Also, the space region 31 formed at an upper portion of the collection unit 30 can have a predetermined gas sealed therein. By thus having a predetermined gas sealed in the space region 31 of the collection unit 30, the gas dissolved in the droplets 2 and ejected into the UFB generation device 20 can have gas exchange with the predetermined gas inside the collection unit 30. Note that in the event of sealing the gas into the collection unit 30, it is preferable to introduce the predetermined gas into the space region 31 of the collection unit 30 and keep the inside of the collection unit 30 at a positive pressure because the gas exchange in the collection unit 30 can then be promoted.

### (Raw Materials of the UFB-Containing Liquid)

### <Gas Component of the Gas-Dissolved Liquid>

The supply unit 10 generates a gas-dissolved liquid in which a gas is dissolved in a liquid to be described later and supplies the generated gas-dissolved liquid to the UFB generation device 20. A gas herein refers to air in an environment in which the apparatus is used unless otherwise noted, but is not limited thereto. Examples of a gas usable as a gas to be dissolved in a liquid include oxygen, nitrogen, hydrogen, ozone, helium, carbon dioxide, methane, ethane, propane, butane, chlorine, and a mixture gas of any of these.

### <Liquid Component of the Gas-Dissolved Liquid>

In the present embodiment, the gas-dissolved liquid L10 in which to generate UFBs is such that the main medium contains at least one selected from group A described below.

Group A: a thermally denaturing material, inorganic salts, amino acids and their salts, sugars and their salts, organic polymers and their salts, inorganic polymers and their salts, a dispersion, and an organic liquid.

### <Medium>

Water is used as the main medium of the gas-dissolved liquid L10. The main medium means a medium accounting for 50% by weight or more of the total medium weight. As the water, pure or ultrapure water or water supplied from water pipes through piping may be used. Water obtained by condensing moisture in atmosphere using a Peltier element or the like may also be used. In a case of using degassed water, the degassed water is supplied to the gas dissolving tank 11 placed under a desired gas atmosphere, which allows gas-dissolved water to be generated according to the Henry's law. In an environment open to the atmosphere, water having approximately 8.4 ppm of oxygen dissolved therein in room temperature is generated. Also, in a case where degassed water is supplied to the gas dissolving tank 11 placed in an oxygen atmosphere, a liquid having 45 ppm of oxygen dissolved therein is generated. Also, a liquid derived from a living organism, specifically blood, spinal fluid, or the like, can be used as the liquid used as the gas-dissolved liquid.

### <Added Components>

The liquid having water as the main component described above contains at least one component selected from the following in a dissolved or dispersed state: a thermally denaturing material, inorganic salts, amino acids and their salts, carbohydrates/sugars and their salts, organic polymers and their salts, inorganic polymers and their salts, a dispersion, and an organic liquid.

Examples of the inorganic salts include metallic salts that dissolve in liquid as salt, such as lithium, sodium, and potassium and multivalent metallic salts such as magnesium, calcium, strontium, barium, titanium, vanadium, chrome, manganese, iron, cobalt, nickel, copper, zinc, aluminum, cadmium, indium, and tin. The multivalent metallic salts contain chloride ion, sulfate ion, hydroxide ion, nitrate ion, or the like as an ion pair. The gas-dissolved liquid preferably contains 0.1% by weight or more of the above-described inorganic salt.

Examples of the amide acids and their salts include isoleucine, leucine, valine, histidine, lysine, methionine, tryptophan, phenylalanine, threonine, asparagine, aspartic acid, alanine, arginine, cysteine, cystine, glutamine, glutamic acid, glycine, proline, serine, tyrosine, theanine, ornithine, citrulline, and taurine and their salts. They also include their modified derivatives.

Examples of the carbohydrates/sugars and their salts include polysaccharides such as starch, oligosaccharide, and dextrin, sugar alcohols such as xylitol, erythritol, and sorbitol, and sugars such as sucrose, lactose, glucose, and fructose and their salts. They also include their modified derivatives. In the mode of the present disclosure, the gas-dissolved liquid preferably contains 0.1% by weight or more of the amino acids and their salts and/or the carbohydrates/sugars and their salts described above.

Examples of the thermally denaturing material include physiologically active substances such as various kinds of inhibitors, lipids/fatty acids, antibiotics, and peptide, biopolymers such as cytokine, hormone, growth factor, enzyme, and protein, nucleic acid polymers such as DNA and RNA, and various kinds of polymerizable monomers.

Examples of the organic polymers and their salts include polyethylene glycol, polypropylene glycol, copolymers of polyethylene glycol and polypropylene glycol, polyacrylic acid, polymethacrylic acid, polyvinyl alcohol, polyvinylpyrrolidone, polyvinyl amide, polyamine, polyethylenimine, carboxy vinyl polymer, alginic acid, carboxymethylcellulose, hydroxyethyl cellulose, methylcellulose, hyaluronic acid, and their salts. The examples also include polymer derivatives obtained by modification of a side chain or a part of these.

Examples of the inorganic polymers and their salts include Si-based polymers such as polycarbosilane, polycarbosilazane, perhydropolysilazane, polyorganosilazane, polyorganoborosilazane, polytitanoxane, polyzirconoxane, polyaluminoxane, and polysiloxane and P-based polymers such as polyphosphoric acid.

Examples of the dispersion include inorganic fine particles such as carbon black, fullerene, graphene, carbon nanotubes, titanium oxide, magnetic particles, and apatite and organic fine particles formed of, e.g., pigment, latex, liposome, polylactic acid, polyglycolic acid, copolymers of polylactic acid and polyglycolic acid, polyethylene glycol, polypropylene glycol, copolymers of polyethylene glycol and polypropylene glycol, or cellulose nano fibers. These fine particles are dispersed in a medium by a surfactant, a polymer dispersant, or surface modification.

The liquid having water as the main component may be used as a mixed solvent of an organic liquid and water. Preferable examples of the organic liquid include organic solvents such as alcohols, esters, and ketones, a polymerizable monomer, oil formed of hydrocarbon, siloxane, or the like, and a nonflammable liquid which is a fluorine liquid or a halogen liquid. Also, a dispersion (such as an emulsion or a micelle) in which any of these organic liquids is uniformly dispersed using a dispersant or the like can be used.

There is no particular limitation as to the organic liquid used, but specific examples include the following.

The specific examples include alkyl alcohols with one to four carbons, such as methyl alcohol, ethyl alcohol, n-propyl alcohol, isopropyl alcohol, n-butyl alcohol, sec-butyl alcohol, and tert-butyl alcohol; amides such as N-methyl-2-pyrrolidone, 2-pyrrolidone, 1,3-dimethyl-2-imidazolidinone N,N-dimethylformamide, and N,N-dimethyl acetamide; ketones or ketoalcohols such as acetone and diacetone alcohol; cyclic ethers such as tetrahydrofuran and dioxane; glycols such as ethylene glycol, 1,2-propylene glycol, 1,3-propylene glycol, 1,2-butanediol, 1,3-butanediol, 1,4-butanediol, 1,5-pentanediol, 1,2-hexanediol, 1,6-hexanediol, 3-methyl-1,5-pentanediol, diethylene glycol, triethylene glycol, and thiodiglycol; polyhydric alcohol lower alkyl ethers such as ethylene glycol monomethyl ether, ethylene glycol monoethyl ether, ethylene glycol monobutyl ether, diethylene glycol monomethyl ether, diethylene glycol monoethyl ether, diethylene glycol monobutyl ether, triethylene glycol monomethyl ether, triethylene glycol monoethyl ether, and triethylene glycol monobutyl ether; polyalkylene glycols such as polyethylene glycol and polypropylene glycol; triols such as glycerine, 1,2,6-hexanetriol, and trimethylolpropane; and the like.

The added components above may be used alone or in combination of two or more kinds.

### <Organic Liquid>

As the gas-dissolved liquid L10 in which to generate UFBs, a liquid having an organic liquid as the main medium can be used. The liquid having an organic medium as the main medium refers to a liquid having the organic liquid described above by 50% by weight or more in relation to the total medium weight. The organic liquid preferably includes at least one of a flammable liquid or an inflammable liquid. These may be used in combination. Examples of the flammable liquid include an organic solvent formed of at least one of alkyl alcohols with one to four carbons such as methyl alcohol, ethyl alcohol, n-propyl alcohol, isopropyl alcohol, n-butyl alcohol, sec-butyl alcohol, and tert-butyl alcohol, esters such as methyl butyrate, methyl salicylate, ethyl formate, ethyl acetate, ethyl propionate, ethyl butyrate, and pentyl pentanoate, ketones such as acetone, methyl ethyl ketone, diisobutyl ketone, and cyclohexanone; polymerizable monomers such as acrylic ester monomers, methacrylic acid ester monomers, styrene monomers, and vinylester monomers; oil formed of at least one of hydrocarbon or siloxane; gasoline; kerosene; light oil; naphtha; and fuel oil for jets. The inflammable liquid is preferably a fluorine liquid or a halogen liquid. The organic liquids described above may further contain water or a substance shown in the group A described above in a dissolved or dispersed state. The viscosity of the gas-dissolved liquid L10 described above is preferably 30 mPa·s or below at the temperature of the time of the UFB generation. In a case where the liquid viscosity is higher than 30 mPa·s, a greater resistance acts in the event where the liquid flies and is ejected from the ejection orifices with a minute diameter, which makes it difficult to generate the UFB-containing liquid stably.

### EXAMPLES

Examples (first to thirteenth examples) according to the present disclosure and comparative examples (first to fourth comparative examples) for the examples are described below.

In the first to thirteenth examples, a gas-dissolved liquid different for each example was supplied to the UFB-containing liquid producing apparatus shown in FIG. 1, and the liquid was ejected and collected. Based on the liquid collected, the following were examined and evaluated: the presence/absence of UFBs, the production stability of the UFB-containing liquid, the particle size of UFBs generated, and the like. In the comparative examples, a liquid was ejected and collected using a liquid ejection method different from that in the first example, and the collected liquid was examined and evaluated in the same manner as the first example. Table 1 shows the production conditions and evaluations of the liquids produced for each of the examples and comparative examples.

### <Evaluation of the Production Stability of the UFB-Containing Liquid>

As the production stability of each of the UFB-containing liquids, whether the UFB-containing liquid could be produced continuously was determined according to the criteria below based on the amount thereof collected in the collection unit 30. Note that the amount collected was measured by measurement of the weight. In the column for the production stability in Table 1, ∘ and x denote the followings states.

∘: The amount collected per minute after 30 minutes is within ±10% of the amount collected per minute initially (the production stability is good).

x: The amount collected per minute after 30 minutes is beyond ±10% of the amount collected per minute initially (the production stability is poor).

### <UFB Detection Method>

As the UFB detection, liquid yet to be ejected from the UFB generation device 20 and liquid ejected and collected by the collection unit 30 were radiated with laser light (wavelength: 532 nm) from a laser pointer, and the intensity of the trajectory was examined. In a case where the intensity of the trajectory of the laser light applied to the ejected liquid is stronger, it means that there is a possibility that UFBs have been generated. Then after that, it was examined whether there was no trajectory of laser light after application of ultrasound for 30 minutes to the sample in which the trajectory was observed. In a case where there was no trajectory of laser light after the 30-minute application of ultrasound, it means that the generated UFBs were lost by the ultrasound. By contrast, in a case where the trajectory of the laser light was still observed after the 30-minute application of ultrasound, it means that the trajectory was attributable not to UFBs, but to scattering of fine particles due to contamination or the like. In the column for UFB detection in FIG. 1, ∘ and x denote the followings states.

∘: The trajectory intensity increases after passing through the UFB generation device and is decreased by ultrasound radiation (UFB generation is good).

x: The trajectory intensity does not change before and after the liquid passes through the UFB generation device or is not decreased by ultrasound radiation (UFB generation is poor).

### <Detection of UFB Particle Size>

A measuring device (model no. SALD-7500) manufactured by Simadzu Corporation was used to measure the average volume particle size of fine bubbles.

### First Example

The gas-dissolved liquid L10 was fabricated by dissolving 0.1% by weight of magnesium sulfate MgSO₄ (manufactured by Kishida Chemical Co., Ltd.) as an additive into ultrapure water as the main medium, and then dissolving air in that solution by bubbling until saturation. The gas-dissolved liquid L10 thus fabricated was supplied to the UFB generation device 20 of the UFB-containing liquid producing apparatus 100 shown in FIG. 1 to produce the UFB-containing liquid L20. Note that in the UFB generation device 20 of the present example, using a printhead with piezoelectric elements (KJ4B-QA manufactured by KYOCERA Corporation), the UFB-containing liquid L20 was produced by ejecting droplets from the ejection orifices 26 using pressure applied to the gas-dissolved liquid by deformation of the piezoelectric elements and collecting the ejected droplets. This liquid producing method is denoted as “PJ” in Table 1. A drive pulse of a voltage of 24 V and a drive frequency of 1 kHz was applied to each of the piezoelectric elements 28 corresponding to 630 ejection orifices 26 of the printhead to eject 12 μL of a droplet 2 from each of the ejection orifices 26. The ejected droplets 2 were collected by the collection unit 30, and the presence of UFBs was examined using the above-described UFB detection method. Also, the particle size of the generated UFBs was measured using the measurement device described above.

### Second to Seventh Examples

In the second to seventh examples, the gas-dissolved liquids L10 having the compositions described in Table 1 were fabricated, and the UFB-containing liquids L20 were produced using the same method as the first example.

Specifically, in the second to seventh examples, the following gas-dissolved liquids L10 were fabricated.
In the second example, the gas-dissolved liquid L10 was fabricated by dissolving 0.1% by weight of calcium chloride as an addition material into ultrapure water as the main medium, and then dissolving air in that solution by bubbling until saturation.
In the third example, the gas-dissolved liquid L10 was fabricated by dissolving 0.5% by weight of L-glutamine as an addition material into ultrapure water as the main medium, and then dissolving air in that solution by bubbling until saturation.
In the fourth example, the gas-dissolved liquid L10 was fabricated by dissolving 0.5% by weight of L-leucine as an addition material into ultrapure water as the main medium, and then dissolving air in that solution by bubbling until saturation.
In the fifth example, the gas-dissolved liquid L10 was fabricated by dissolving 1.0% by weight of D-glucose as an addition material into ultrapure water as the main medium, and then dissolving air in that solution by bubbling until saturation.
In the sixth example, the gas-dissolved liquid L10 was fabricated by dissolving 1.0% by weight of albumin as an addition material into ultrapure water as the main medium, and then dissolving air in that solution by bubbling until saturation.
In the seventh example, the gas-dissolved liquid L10 was fabricated by dissolving 0.01% by weight of streptomycin as an addition material into ultrapure water as the main medium, and then dissolving air in that solution by bubbling until saturation.

Using the gas-dissolved liquids L10 fabricated as above, the droplets 2 were ejected and collected, and based on the liquid collected, the examination and evaluation were conducted in the same manner as the first example.

### Eighth Example

In the eighth example, the gas-dissolved liquid L10 was fabricated by mixing ethanol (30% by volume) as other medium into ultrapure water (70% by volume) as the main medium, and then dissolving air in that mixture of media by bubbling until saturation. Then, using the gas-dissolved liquid L10 thus fabricated, the droplets 2 were ejected and collected using the same method as the first example, and based on the liquid L20 collected, the examination and evaluation were conducted in the same manner as the first example.

### Ninth Example

In the ninth example, the gas-dissolved liquid L10 was fabricated by mixing water (30% by volume) as other medium into ethanol (70% by volume) as the main medium, and then dissolving air in that mixture of media by bubbling until saturation. Then, using the gas-dissolved liquid L10 thus fabricated, the droplets 2 were ejected and collected using the same method as the first example, and based on the liquid L20 collected, the examination and evaluation were conducted in the same manner as the first example. In the present example, a drive pulse of a voltage of 24 V and a drive frequency of 1 kHz was applied to each of the piezoelectric elements 28 corresponding to 630 ejection orifices 26 of the printhead to eject 14 μL of a droplet 2 from each of the ejection orifices 26.

### Tenth Example

In the tenth example, the gas-dissolved liquid L10 was fabricated by dissolving air into kerosene as the main medium by bubbling until saturation. Then, the gas-dissolved liquid L10 thus fabricated was ejected as droplets 2 and collected using the same method as the first example. After that, based on the collected liquid L20, examination and evaluation were conducted in the same manner as the first example. Note that in the present example, the piezoelectric elements 28 were driven in the same manner as the ninth example, and 14 μL of droplet 2 was ejected from each of the ejection orifices 26.

### Eleventh Example

In the eleventh example, the gas-dissolved liquid was fabricated by dissolving air into ultrapure water as the main medium by bubbling until saturation, and then dissolving 0.03% by weight of yeast DNA as an additive therein. The gas-dissolved liquid thus fabricated was supplied to a Labojet-600 (manufactured by MicroJet Inc.), a drive voltage and a drive pulse to apply to the piezoelectric elements 28 were adjusted, and 12 μL of droplets 2 were ejected. The ejected droplets 2 were collected by the collection unit 30, and the collected liquid L20 was examined and evaluated in the same manner as the first example.

### Twelfth Example

Titanium oxide fine particles (STS-21 manufactured by ISHIHARA SANGYO KAISHA, LTD.) by 4.5% by weight in solid content concentration and a polymer dispersant (T-50 manufactured by TOAGOSEI CO., LTD.) by 0.5% by weight in solid content concentration were added to ultrapure water as the main medium and dispersed using an ultrasonic homogenizer. Air was dissolved in this dispersion liquid by bubbling until saturation to fabricate the gas-dissolved liquid L10. The gas-dissolved liquid L10 thus fabricated was ejected as the droplets 2 and collected in the same manner as the first example. In a case where the collected liquid L20 contained fine particles, the fine particles were removed using an ultracentrifuge, and the supernatant liquid was examined and evaluated in the same manner as the first example.

### Thirteenth Example

The gas-dissolved liquid L10 was fabricated using a culture medium for mammalian cells (manufactured by Expression Systems, LLC) as the main medium and dissolving air in this by bubbling until saturation. The gas-dissolved liquid L10 thus fabricated was ejected as droplets 2 and collected in the same manner as the first example. The collected liquid L20 was examined and evaluated in the same manner as the first example.

### First Comparative Example

In the first comparative example, a UFB generation device with a different configuration from the examples described above was used. The UFB generation device used had ten boards mounted side by side in series in and along the flow channel in which the liquid flows, each board having 10,000 heaters disposed thereon. The same gas-dissolved liquid as that in the first example was supplied to the flow channel, and the heaters were driven by application of pulse signals at a drive frequency of 20 kHz. The liquid flowed over the heaters at a flow rate of 1.0 L/h. The liquid that passed over the heaters was collected, and the collected liquid was examined and evaluated in the same manner as the first example.

### Second Comparative Example

The same gas-dissolved liquid as that in the first comparative example was used in the second comparative example. An inkjet head was used as the UFB generation device, the inkjet head being configured to generate air bubbles in a liquid by heating the liquid with heat generation elements and eject droplets from the ejection orifices using pressure generated upon the generation of the air bubbles. The droplets ejected from the inkjet head were collected. Note that this liquid producing method is denoted as “BJ” in Table 1. The collected liquid was examined and evaluated in the same manner as the first example.

### Third Comparative Example

In the third comparative example, the same gas-dissolved liquid as that in the ninth example was fabricated, the fabricated gas-dissolved liquid was supplied to the same UFB generation device as that in the first comparative example, and the liquid that flowed over the heaters was collected. The conditions for the driving of the heaters and the flow rate of the liquid were the same as those in the first comparative example. The collected liquid was examined and evaluated in the same manner as the first example.

### Fourth Comparative Example

In the fourth comparative example, the gas-dissolved liquid fabricated in the same manner as that in the first example was supplied to a shower head (product name: Bollina, manufactured by TKS.) that produces a fine-bubble-containing liquid using swirling flows, and the showered liquid ejected from the shower head was collected into a container. The collected liquid was examined and evaluated in the same manner as the first example.

Table 1 below shows results of the examination of the examples and comparative examples in terms of the UFB generation stability, the presence/absence of UFBs, and the particle size thereof.

TABLE 1

| | | | | | | | Liquid | | | Average |
|---|---|---|---|---|---|---|---|---|---|---|
| | Main | Volume | Other | Volume | addition | Mass | production | Production | UFB | volume |
| | medium | % | medium | % | material | % | method | stability | detection | particle size |
| 1st Example | water | 100 | — | — | MgSO₄ | 0.1 | PJ | ○ | ○ | 230 |
| 2nd Example | water | 100 | — | — | CaCl₂ | 0.1 | PJ | ○ | ○ | 230 |
| 3rd Example | water | 100 | — | — | L-glutamine | 0.5 | PJ | ○ | ○ | 250 |
| 4th Example | water | 100 | — | — | L-leucine | 0.5 | PJ | ○ | ○ | 220 |
| 5th Example | water | 100 | — | — | D-glucose | 1.0 | PJ | ○ | ○ | 215 |
| 6th Example | water | 100 | — | — | albumin | 1.0 | PJ | ○ | ○ | 340 |
| 7th Example | water | 100 | — | — | streptomycin | 0.01 | PJ | ○ | ○ | 150 |
| 8th Example | water | 70 | ethanol | 30 | — | — | PJ | ○ | ○ | 195 |
| 9th Example | ethanol | 70 | water | 30 | — | — | PJ | ○ | ○ | 205 |
| 10th Example | kerosene | 100 | — | — | — | — | PJ | ○ | ○ | 120 |
| 11th Example | water | 100 | — | — | yeast DNA | 0.03 | PJ | ○ | ○ | 250 |
| 12th Example | water | 100 | — | — | titanium oxide | 5.0 | PJ | ○ | ○ | 210 |
| | | | | | fine particles | | | | | |
| | | | | | and dispersant | | | | | |
| 13th Example | culture | 100 | — | — | — | — | PJ | ○ | ○ | 180 |
| | medium | | | | | | | | | |
| 1st Comparative | water | 100 | — | — | MgSO₄ | 0.1 | pass over | ○ | x | — |
| Example | | | | | | | heaters | | | |
| 2nd Comparative | water | 100 | — | — | MgSO₄ | 0.1 | BJ | x | — | — |
| Example | | | | | | | | | | |
| 3rd Comparative | ethanol | 70 | water | 30 | — | — | pass over | ○ | x | — |
| Example | | | | | | | heaters | | | |
| 4th Comparative | water | 100 | — | — | MgSO₄ | 0.1 | swirl flow | ○ | ○ | 600 |
| Example | | | | | | | method | | | |

As shown in Table 1, in the first to thirteenth examples, it was observed that the collected liquid had UFBs with small particular size generated therein substantially uniformly. Also, in the first to thirteenth examples, the UFB-containing liquid was produced stably, and no degradation in production stability due to passage of time was observed.

By contrast, in the first comparative example, the liquid was collected, but there were no UFBs generated therein. A reason for this can be because the heat generated by the heaters caused what is called kogation, which is buildup of deposits on the heaters, and no pressure change was caused by generation of bubbles.

In the second comparative example as well, droplets could not be ejected due possibly to kogation, and the UFB-containing liquid could not be produced stably.

In the third comparative example, film boiling did not properly occur on the heaters in the medium mixture of ethanol and water, which is a possible reason why no UFBs were generated.

In the fourth comparative example, UFBs were generated. However, their average volume particle size was large, and also air bubbles (micro bubbles or milli-bubbles) larger than UFBs and the like were also mixed therein, which can be a reason why it is not suitable for long-term storage.

### OTHER EMBODIMENTS

In the above embodiment, the piezoelectric elements 28 were used as pressure application units to apply pressure to liquid to eject droplets from the ejection orifices 26 with a minute diameter formed in the ejection orifice forming member 25, but the present invention is not limited to this. It is also possible to eject droplets from the ejection orifices by applying ultrasonic vibration to the plate-shaped ejection orifice forming member 25 shown in the above embodiment and applying pressure to the liquid in the flow channel using the ultrasonic vibration. In this case, the ejection orifice forming member 25 is the unit that applies pressure to the liquid. It is also possible to employ a configuration where droplets are ejected from the ejection orifices by applying pressure to the liquid in the flow channel using, as a pressure application unit, a plate-shaped pressure application member facing the ejection orifice forming member.

The present disclosure can provide an apparatus for generating an UFB-containing liquid and a method for generating an UFB-containing liquid that are capable of stably generating a UFB-containing liquid which has high concentration and can be stored for a long period of time.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A method for producing an ultrafine-bubble-containing liquid, the method comprising:
supplying a liquid containing at least one substance selected from a group stated below to a liquid supply region where an ejection orifice forming member in which an ejection orifice with a minute diameter is formed is disposed; and
ejecting the liquid from the ejection orifice as a droplet by causing a pressure application unit in contact with the liquid to apply pressure to the liquid supplied to the liquid supply region,
the group consisting of a thermally denaturing material, inorganic salts, amino acids and their salts, carbohydrates/sugars and their salts, organic polymers and their salts, inorganic polymers and their salts, a dispersion, and an organic liquid.

2. The method for producing an ultrafine-bubble-containing liquid according to claim 1, wherein
the thermally denaturing material is at least one selected from a physiologically active substance, a nucleic acid polymer, a biopolymer, and a polymerizable monomer.

3. The method for producing an ultrafine-bubble-containing liquid according to claim 2, wherein
the physiologically active substance is an inhibitor, a lipid, a fatty acid, an antibiotic, or a peptide,
the biopolymer is cytokine, hormone, a growth factor, enzyme, or protein, and
the nucleic acid polymer is DNA or RNA.

4. The method for producing an ultrafine-bubble-containing liquid according to claim 1, wherein
the dispersion is at least one of inorganic fine particles or organic fine particles.

5. The method for producing an ultrafine-bubble-containing liquid according to claim 4, wherein
the inorganic fine particles are fine particles of carbon black, fullerene, graphene, carbon nanotubes, titanium oxide, magnetic fine particles, or apatite, and
the organic fine particles are fine particles of pigment, latex, liposome, polylactic acid, polyglycolic acid, a copolymer of polylactic acid and polyglycolic acid, polyethylene glycol, polypropylene glycol, a copolymer of polyethylene glycol and polypropylene glycol, or cellulose nano fibers.

6. The method for producing an ultrafine-bubble-containing liquid according to claim 1, wherein
the organic liquid contains at least one of a flammable liquid or an inflammable liquid.

7. The method for producing an ultrafine-bubble-containing liquid according to claim 6, wherein
the flammable liquid is at least one alcohol selected from methyl alcohol, ethyl alcohol, and isopropyl alcohol, a polymerizable monomer, oil formed of at least one of hydrocarbon or siloxane, gasoline, kerosene, light oil, naphtha, or fuel oil for jets, and
the inflammable liquid is a fluorine liquid or a halogen liquid.

8. The method for producing an ultrafine-bubble-containing liquid according to claim 1, wherein
the liquid has a viscosity of 30 mPa·s.

9. The method for producing an ultrafine-bubble-containing liquid according to claim 1, further comprising dissolving a predetermined gas in the liquid.

10. The method for producing an ultrafine-bubble-containing liquid according to claim 1, wherein
in the ejecting, the liquid is ejected as droplets from a plurality of the ejection orifices formed in the ejection orifice forming member.

11. The method for producing an ultrafine-bubble-containing liquid according to claim 1, wherein
a plurality of the pressure application units are provided facing a plurality of the ejection orifices respectively, and
in the ejecting, each of the plurality of pressure application units applies pressure to the liquid to eject the liquid as droplets from the plurality of ejection orifices.

12. The method for producing an ultrafine-bubble-containing liquid according to claim 1, wherein
the ejection orifice is 100 μm or smaller in diameter.

13. The method for producing an ultrafine-bubble-containing liquid according to claim 1, wherein
the pressure application unit is formed by a piezoelectric element which deforms upon application of voltage, and
in the ejecting, the piezoelectric element applies pressure to the liquid in the liquid supply region by deforming, so that the liquid is ejected from the ejection orifice as a droplet.

14. The method for producing an ultrafine-bubble-containing liquid according to claim 1, further comprising collecting the liquid ejected from the ejection orifice.

15. An apparatus for producing an ultrafine-bubble-containing liquid, the apparatus comprising:
an ejection orifice forming member in which an ejection orifice with a minute diameter is formed and which is disposed at a liquid supply region to which a liquid containing at least one substance selected from a group stated below is supplied; and
a pressure application unit that causes the liquid to be ejected from the ejection orifice as a droplet by being in contact with the liquid and applying pressure to the liquid supplied to the liquid supply region,
the group consisting of a thermally denaturing material, inorganic salts, amino acids and their salts, carbohydrates/sugars and their salts, organic polymers and their salts, inorganic polymers and their salts, a dispersion, and an organic liquid.

16. The apparatus for producing an ultrafine-bubble-containing liquid according to claim 15, further comprising a gas dissolution unit that dissolves a predetermined gas in the liquid.

17. The apparatus for producing an ultrafine-bubble-containing liquid according to claim 15, wherein
a plurality of the ejection orifices are formed in the ejection orifice forming member, and
the pressure application unit applies pressure to the liquid to eject the liquid as droplets from the plurality of ejection orifices.

18. The apparatus for producing an ultrafine-bubble-containing liquid according to claim 15, wherein
a plurality of the pressure application units are provided facing a plurality of the ejection orifices respectively, and
each of the plurality of pressure application units applies pressure to the liquid to eject the liquid as droplets from the plurality of ejection orifices.

19. The apparatus for producing an ultrafine-bubble-containing liquid according to claim 15, wherein
the ejection orifice is 100 μm or smaller in diameter.

20. The apparatus for producing an ultrafine-bubble-containing liquid according to claim 15, wherein
the pressure application unit is formed by a piezoelectric element which deforms upon application of voltage, and
the piezoelectric element applies pressure to the liquid in the liquid supply region by deforming, so that the liquid is ejected from the ejection orifice as a droplet.

21. The apparatus for producing an ultrafine-bubble-containing liquid according to claim 15, further comprising a collection unit that collects the liquid ejected from the ejection orifice.
